(19) United States
(12) Patent Application Publication
(10) Pub. No.: US 2017/0135641 A1
(43) Pub. Date: May 18, 2017

(54) METHOD OF MANUFACTURING MULTIPLE MICROELECTRODES ON NEEDLE AND NEEDLE MANUFACTURED BY THE SAME

(71) Applicant: GWANGJU INSTITUTE OF SCIENCE AND TECHNOLOGY, Gwangju (KR)

(72) Inventors: Jong-Hyun LEE, Gwangju (KR); Joho YUN, Gwangju (KR); Hyeon Woo KIM, Gwangju (KR); Yangkyu PARK, Gwangju (KR)

(21) Appl. No.: 15/336,820

(22) Filed: Oct. 28, 2016

(30) Foreign Application Priority Data
Nov. 12, 2015 (KR) 10-2015-0159186

(51) Int. Cl. A61B 5/00; A61N 1/05; A61B 5/04

(52) U.S. Cl. CPC A61B 5/6848; A61B 5/04; A61N 1/0502; A61B 2562/125

(57) ABSTRACT

Disclosed herein are a method of manufacturing multiple microelectrodes on a syringe needle and a syringe needle manufactured by the same. The syringe needle includes multiple interdigitated electrodes (IDEs) placed on a surface of a portion of the syringe needle spaced apart from a tip of the syringe needle, a pair of interconnection lines for electrical connection of the multiple IDEs, the interconnection lines electrically connecting a first group of the IDEs on the left and a second group of the IDEs on the right through one end of each of the interconnection lines, and a conductor for electrical connection to a PCB provided to a main body of a syringe at the other end of each of the interconnection lines, wherein the multiple IDEs are basically formed of a material for a dielectric layer and are alternately arranged at a first distance from one another.

### BACKGROUND

1. Technical Field

The present invention relates to a method of manufacturing multiple microelectrodes on a syringe needle, and a syringe needle manufactured by the same.

2. Description of the Related Art

Conventionally, a syringe needle has been used to extract blood from the skin or to inject a medicine into the body. A recent syringe needle has neural microelectrodes to detect electrical signals from normal tissue and pathological tissue, thereby making a diagnosis based on a difference between the electrical signals. In addition, a syringe needle is also used to transmit electrical stimulation to a body tissue or to provide therapeutic effects using electrical resistance heat.

When such a syringe needle having a non-flat tip, it is difficult to fabricate through photolithography due to incomplete connection or a step between a photoresist and a photomask during a manufacturing process, interference during exposure to UV light, and the like.

In the related art, U.S. Patent Publication No. 2010-0076534 discloses a soft needle tip structure which has at least three individual electrodes to facilitate introduction of an electrical stimulation lead for peripheral nerve stimulation. However, this needle tip structure has a limitation in expansion of an electrode structure, and is difficult to apply to a syringe needle having a pointed tip.

In addition, “Direct metal micropatterning on needle-type structures towards bioimpedance and chemical sensing applications” (December 2014, Journal of Micromechanics and Microengineering) discloses a method in which a screen mesh with an electrode pattern is placed on a syringe needle, followed by application of a liquid metal, and the liquid metal is primarily transferred to the syringe needle using a squeegee, followed by electroplating subsequent to drying, thereby fabricating a final electrode. However, this method has a limitation in simplifying and generalizing a manufacturing process of a syringe needle having multiple microelectrodes in that it is difficult to fabricate or replace a mask; the mask must be cleaned in each process; and it is difficult to reduce the size of the electrodes on the syringe needle.

### BRIEF SUMMARY

The present invention has been conceived to solve such problems in the art and it is an aspect of the present invention to provide a method of manufacturing multiple microelectrodes on a tip of a syringe needle having a diameter of several hundred micrometers, and a syringe needle manufactured by the same.

It is another aspect of the present invention to provide a method of manufacturing multiple microelectrodes on a tip of a syringe needle which can increase yield of photoresist coating required for photolithography of a thin conical syringe needle and can form multiple microelectrodes on the tip of the syringe needle without a need for a separate flexible polymer thin film photomask, thereby further simplifying a manufacturing process beyond typical methods.

In accordance with one aspect of the present invention, there is provided a method of manufacturing multiple microelectrodes on a syringe needle. The method is aimed at forming multiple microelectrodes on a surface of a portion of a syringe needle spaced apart from a tip of the syringe needle, and includes: coating a first dielectric layer on the surface; depositing a metal layer on the surface coated with the dielectric layer by sputtering; depositing a photoresist layer on the surface coated with the metal layer by spray coating; exposing the surface coated with the photoresist layer to UV light using a flexible film mask; leaving the photoresist layer for a predetermined period of time, followed by wet etching the metal layer; and removing the photoresist layer and may further include additionally coating a second dielectric layer after removal of the photoresist layer.

Each of the first dielectric layer and the second dielectric layer may be formed of a synthetic polymer compound and a nonmetallic dielectric material, and the synthetic polymer compound may be any one selected from Parylene C, Teflon, PET, and polyimide, and the nonmetallic dielectric material may be an oxide or a nitride having high melting point.

The portion of the syringe needle having the multiple microelectrodes formed thereon may extend from a point at a distance of 300 μm to 500 μm (preferably 390 μm) from the tip of the syringe needle to a point at a distance of 1,000 μm to 1,200 μm from the tip of the syringe needle.

In accordance with another aspect of the present invention, there is provided a syringe needle having microelectrodes on a surface thereof, wherein the microelectrodes are formed by the method as set forth above. The syringe needle includes; multiple interdigitated electrodes (IDEs) placed on a surface of a portion of the syringe needle spaced apart from a tip of the syringe needle and formed of a first metal; and a pair of interconnection lines for electrical connection of the multiple IDEs, the interconnection lines electrically connecting a first group of the IDEs on the left and a second group of the IDEs on the right through one end of each of the interconnection lines, wherein the multiple IDEs are basically formed of a material for a dielectric layer and are alternately arranged at a first distance from one another.

Each of the multiple IDEs may be formed by a process including: coating a dielectric layer; depositing a metal layer by sputtering; depositing a photoresist layer by spray coating; exposing the photoresist layer to UV light using a flexible film mask; wet etching the metal layer, and removing the photoresist layer.

The syringe needle may further include: a conductor for electrical connection to a PCB provided to a main body of a syringe at the other end of each of the interconnection lines, wherein the conductor may be any one selected from a metal wire, a conductive tape, and a flexible electrode.

A surface of the syringe needle, on which the multiple IDEs, the interconnection lines and the conductors are placed, may be coated with a dielectric layer; the dielectric layer may be formed of a synthetic polymer compound and a nonmetallic dielectric material; and the synthetic polymer compound may be any one selected from Parylene C, Teflon, PET, and polyimide.

The syringe needle may have a diameter of 700 μm, and the interconnection lines are respectively formed at equidistant points at an angle of 65.5° from the center of the syringe needle such that the pair of interconnection lines has a width of 400 μm. Each of the IDEs may have a width of 25 μm, and the IDEs of the first IDE group may be spaced a first distance of 35 μm from the respective IDEs of the second IDE group.

In the aspects of the present invention, the metal layer may be formed of any one selected from chromium (Cr), gold (Au), platinum (Pt), copper (Cu), silver (Ag), palladium (Pd), and a mixture thereof, and the flexible film mask may be a polyester mask.

According to the present invention, since a flexible polymer thin film photomask does not need to be separately prepared, it is possible to reduce process time and costs. In addition, a highly durable polyester mask can be repeatedly used in a process of manufacturing a syringe needle having multiple microelectrodes.

Further, according to the present invention, application of a photoresist can be performed at the same time as heating of a syringe needle, thereby improving overall coating and fabrication yields.

Thus, according to the present invention, it is possible to simplify a manufacturing process, thereby allowing mass production.

### BRIEF DESCRIPTION OF THE DRAWINGS

The above and other aspects, features, and advantageous effects of the present invention will become apparent from the detailed description of the following embodiments in conjunction with the accompanying drawings, in which;

FIG. 1 is a schematic view illustrating a typical method for fabricating a syringe needle having microelectrodes;

FIG. 2 is a schematic view illustrating a method for fabricating a syringe needle having microelectrodes according to the present invention;

FIG. 3 is a flowchart of the method shown in FIG. 2;

FIG. 4 is a schematic view of a syringe needle having multiple microelectrodes fabricated by the method according to the present invention;

FIG. 5 is a plan view of the syringe needle shown in FIG. 4; and

FIG. 6 shows the syringe needle according to the present invention in actual use.

### DETAILED DESCRIPTION

The particular structural or functional descriptions of embodiments according to the concepts of the present invention disclosed in the specification or the application are only intended for the purpose of describing embodiments according to the concepts of the present invention and the embodiments according to the concepts of the present invention may be practiced in various forms and should not be construed as being limited to those described in the specification or the application.

The present invention may be realized by various embodiments, and some exemplary embodiments of the present invention will be described in detail with reference to the accompanying drawings. However, it should be understood that the present invention is not limited to the following embodiments, and that various modifications, substitutions, and equivalent embodiments can be made by those skilled in the art without departing from the spirit and scope of the present invention.

Although the terms “first”, “second”, etc. may be used herein to describe various elements, components, regions, layers, and/or sections, these elements, components, regions, layers, and/or sections should not be limited by these terms. These terms are used to distinguish one element, component, region, layer, and/or section from another element, component, region, layer, and/or section. Thus, a first element, component, region, layer, and/or section discussed below could be termed a second element, component, region, layer, and/or section without departing from the teachings of the present disclosure.

The terminology used herein is for the purpose of describing particular embodiments and is not intended to be limitative. As used herein, the singular forms, “a,” “an,” and “the” are intended to include the plural forms as well, unless context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined herein, all terms including technical or scientific terms used herein have the same meanings as commonly understood by those skilled in the art to which the present invention pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of a method of manufacturing multiple microelectrodes on a syringe needle according to the present invention and a syringe needle manufactured by the same will be described in detail.

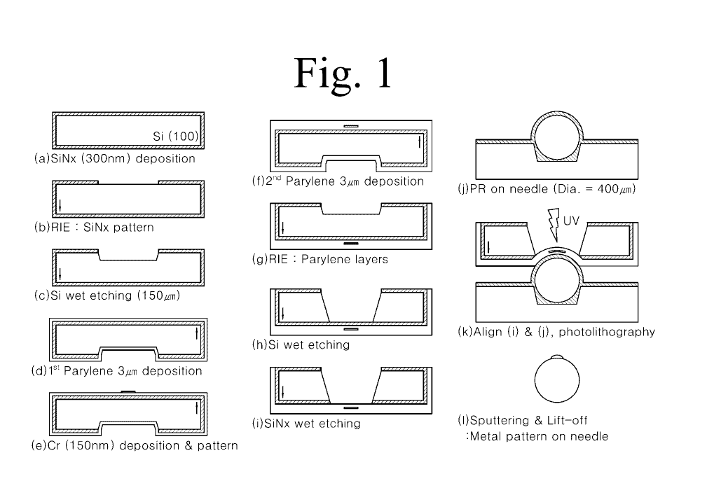

FIG. 1 is a view illustrating a typical method of manufacturing a syringe needle having microelectrodes. The typical method requires at least 20 steps, including: deposition of SiNx on a silicon substrate (step A); reactive ion etching (RIE) for SiNx patterning (step B); silicon wet etching (step C); primary deposition of Parylene to a thickness of 3 μm (step D), chromium deposition and patterning (step E); secondary Parylene deposition (step F); RIE of Parylene layers (step G); silicon wet etching (step H); SiNx wet etching (step I); Coating of a photoresist (PR) on a needle (step J); alignment of a structure obtained after step I with a structure obtained by step J and photolithography (step K); and sputtering and lift-off (step L).

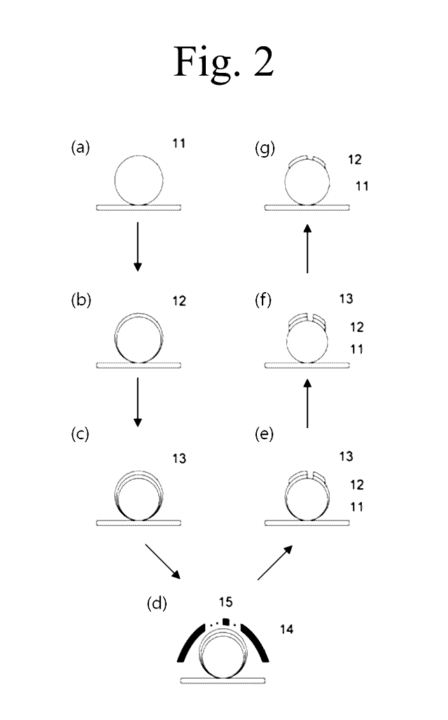

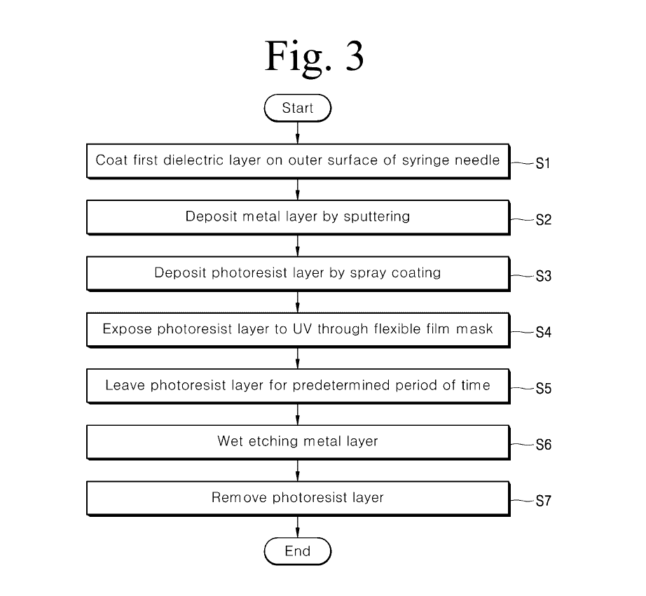

FIG. 2 is a view illustrating a method of manufacturing a syringe needle having microelectrodes according to the present invention, and FIG. 3 is a flowchart of the method of manufacturing a syringe needle having microelectrodes according to the present invention.

First, a dielectric layer 11 is coated onto an outer surface of a needle (FIG. 2(a), S10 in FIG. 3). The dielectric layer is formed of a synthetic polymer compound and a nonmetallic dielectric material. The synthetic polymer compound may be any one selected from among Parylene C, Teflon, PET, and polyimide, and the nonmetallic dielectric material may be an oxide or a nitride having high melting point. If the dielectric layer is only composed of the synthetic polymer compound without the nonmetallic dielectric material, the dielectric layer can lose insulation properties when the melting point of a wire is reached during wire bonding for electrically connecting the syringe needle to a printed circuit board (PCB). Thus, the dielectric layer needs to be formed of the synthetic polymer compound and the nonmetallic dielectric material. It should be understood that the nonmetallic dielectric material has a higher melting point than a metal layer described below.

Preferably, the synthetic polymer compound is Parylene C. Coating of the Parylene is a process in which a powdery raw material is vaporized under a vacuum to form a polymeric film and may be performed by chemical vapor deposition (CVD).

Then, a metal layer 12 is formed on the surface of the needle coated with the Parylene by sputtering (FIG. 2(b), S20 in FIG. 3). The metal layer may be any one selected from chromium (Cr), gold (Au), platinum (Pt), copper (Cu), silver (Ag), palladium (Pd), and a mixture thereof. Here, a ratio between metal components of the mixture may be variable. Although the metal layer has been described as being formed of the above metals, it should be understood that the metal layer may be formed of any suitable metal so long as the metal is suitable for electrochemical sensing and can be deposited by electroplating, evaporation, or sputtering.

Then, a photoresist layer 13 is formed on the surface of the needle coated with the metal layer by spray coating (FIG. 2 (c), S30 in FIG. 3).

Thereafter, the surface of the needle coated with the photoresist layer is exposed to UV light 15 at a portion thereof on which microelectrodes are to be formed using a flexible film mask 14 (FIG. 2(d), S40 in FIG. 3). The flexible film mask may be, for example, a polyester mask. In a typical method, a polymer photomask must be separately fabricated. Conversely, in the method according to the present invention, the film mask in S40 may be repeatedly used, whereby a limitation in mass production can be overcome.

Then, after the photoresist is allowed to stand for a predetermined period of time (FIG. 2(e), S50 in FIG. 3), the metal layer is subjected to wet etching (FIG. 2(f), S60 in FIG. 3). Thereafter, the photoresist layer is removed (FIG. 2(g), S70 in FIG. 3), whereby the microelectrodes can be formed on the surface of the needle. Although not shown in FIGS. 2 and 3, a dielectric layer may be additionally coated onto the surface of the needle after removal of the photoresist layer.

The method according to the present invention described with reference to FIGS. 2 and 3 allows multiple microelectrodes to be formed on a tip of a syringe needle through only 7 steps, thereby simplifying a manufacturing process. In addition, in the method according to the present invention, application of a photoresist can be performed at the same time as heating of the syringe needle, thereby shortening the time required for soft baking and improving overall coating and fabrication yields.

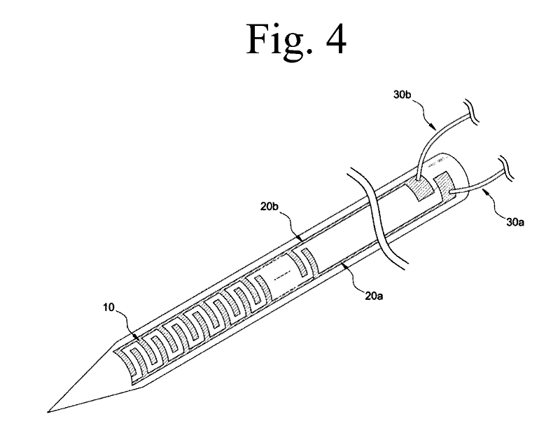

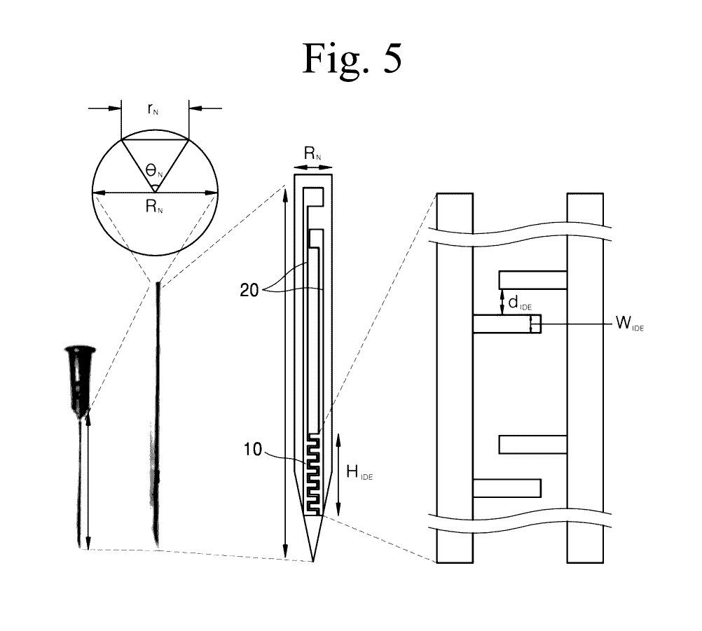

Next, a syringe needle having multiple microelectrodes fabricated by the method of the present invention will be described with reference to FIGS. 4 and 5.

FIG. 4 is a schematic view of a syringe needle having multiple microelectrodes fabricated by the method of the present invention, and FIG. 5 shows the syringe needle in a plan view for convenience of description.

The syringe needle according to the present invention is composed of multiple interdigitated electrodes (IDEs) 10 and interconnection lines 20a, 20b for electrical connection of the IDEs. The multiple IDEs 10 are microelectrodes and may be fabricated by the method described with reference to FIGS. 2 and 3. The IDEs 10 are sensing electrodes which can transmit/receive electrical stimulation to/from a body tissue, and the electrical stimulation is transmitted to the body tissue through the interconnection line 20.

The IDEs 10 are placed on a surface of a portion of the syringe needle spaced apart from a tip of the needle. For example, the IDEs may be placed from a point separated a distance of 300 μm to 500 μm from the tip of the syringe needle to a point at a distance of 1,000 μm to 1,200 μm from the tip of the syringe needle (the section HIDE of FIG. 5). The multiple IDEs are basically formed of a material for the dielectric layer (i.e. Parylene C), and are alternately arranged at a first distance from one another.

Specifically, referring to FIG. 5(c), each of the IDEs may have a width (WIDE) of 20 μm to 30 μm (preferably, 25 μm), and a distance (d_{IDE}) between adjacent IDEs may be greater than the width (W_{IDE}), and is preferably 35 μm.

The interconnection line for electrical connection of the multiple IDEs may include a pair of interconnection lines including a first interconnection line 20a for a first group of IDEs placed on the left and a second interconnection line 20b for a second group of IDEs placed on the right. Referring to FIG. 5(a), assuming that a diameter (R_{N}) of the syringe needle is 700 μm, the pair of interconnection lines 20a, 20b may be formed at equidistant points at an angle (θ_{N}) of 65.5° from the center of the syringe needle such that the pair of interconnection lines has a width (r_{N}) of 400 μm.

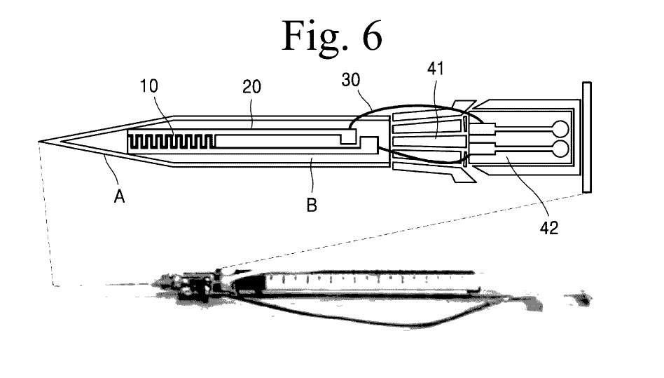

In actual use, the syringe needle may be assembled to a syringe including a PCB, as shown in FIG. 6. In this case, the syringe needle further includes a conductor 30 for electrical connection to the PCB 42, which is placed in close contact with a hub 41 of a main body of the syringe, at an end of the interconnection line opposite an end connected to the IDEs, wherein the conductor 30 may be any one selected from a metal wire, a conductive tape, and a flexible electrode. In the syringe assembly as shown in FIG. 6, both of the region A and the region B are a dielectric layer, and the region A may be subjected to passivation and the region B may be subjected to insulation treatment.

Although some embodiments have been described herein, it should be understood by those skilled in the art that these embodiments are given by way of illustration only, and that various modifications, variations and alterations can be made without departing from the spirit and scope of the invention. Therefore, the embodiments and the accompanying drawings should not be construed as limiting the spirit of the present invention, but should be construed as illustrating the spirit of the present invention. The scope of the invention should be interpreted according to the following appended claims as covering all modifications or variations derived from the appended claims and equivalents thereof.

What is claimed is:

1. A method of manufacturing multiple microelectrodes on a surface of a portion of a syringe needle spaced apart from a tip of the syringe needle, comprising:
coating a first dielectric layer on the surface;
depositing a metal layer on the surface coated with the dielectric layer by sputtering;
depositing a photoresist layer on the surface coated with the metal layer by spray coating;
exposing the surface coated with the photoresist layer to UV light using a flexible film mask;
leaving the photoresist layer for a predetermined period of time, followed by wet etching the metal layer; and
removing the photoresist layer.

2. The method according to claim 1, further comprising:
additionally coating a second dielectric layer after removal of the photoresist layer.

3. The method according to claim 2, wherein each of the first dielectric layer and the second dielectric layer is formed of a synthetic polymer compound and a nonmetallic dielectric material, and the synthetic polymer compound is any one selected from Parylene C, Teflon, PET, and polyimide.

4. The method according to claim 3, wherein the metal layer is formed of any one selected from chromium (Cr), gold (Au), platinum (Pt), copper (Cu), silver (Ag), palladium (Pd), and a mixture thereof.

5. The method according to claim 3, wherein the flexible film mask is a polyester mask.

6. The method according to claim 3, wherein the portion of the syringe needle having the multiple microelectrodes formed thereon extends from a point separated a distance of 300 μm to 500 μm from the tip of the syringe needle.

7. A syringe needle having microelectrodes on a surface thereof, wherein the microelectrodes are formed by the method according to claim 1.

8. A syringe needle, comprising;
multiple interdigitated electrodes (IDEs) placed on a surface of a portion of the syringe needle spaced apart from a tip of the syringe needle and formed of a first metal; and
a pair of interconnection lines for electrical connection of the multiple IDEs, the interconnection lines electrically connecting a first group of the IDEs on the left and a second group of the IDEs on the right through one end of each of the interconnection lines,
wherein the multiple IDEs are basically formed of a material for a dielectric layer and are alternately arranged at a first distance from one another.

9. The syringe needle according to claim 8, wherein each of the multiple IDEs is formed by a process comprising: coating a dielectric layer; depositing a metal layer by sputtering; depositing a photoresist layer by spray coating; exposing the photoresist layer to UV light using a flexible film mask; wet etching the metal layer, and removing the photoresist layer.

10. The syringe needle according to claim 9, further comprising:
a conductor for electrical connection to a PCB provided to a main body of a syringe at the other end of each of the interconnection lines,
wherein the conductor is any one selected from a metal wire, a conductive tape, and a flexible electrode.

11. The syringe needle according to claim 9, wherein a surface of the syringe needle, on which the multiple IDEs, the interconnection lines, and the conductors are placed, is coated with a dielectric layer.

12. The syringe needle according to claim 11, wherein the dielectric layer is formed of a synthetic polymer compound and a nonmetallic dielectric material, and the synthetic polymer compound is any one selected from Parylene C, Teflon, PET, and polyimide.

13. The syringe needle according to claim 9, wherein the metal layer is formed of any one selected from chromium (Cr), gold (Au), platinum (Pt), copper (Cu), silver (Ag), palladium (Pd), and a mixture thereof.

14. The syringe needle according to claim 9, wherein the syringe needle has a diameter of 700 μm, and the interconnection lines are respectively formed at equidistant points at an angle of 65.5° from the center of the syringe needle such that the pair of interconnection lines has a width of 400 μm.

15. The syringe needle according to claim 13, wherein each of the IDEs has a width of 25 μm, and the IDEs of the first IDE group are spaced a first distance of 35 μm from the respective IDEs of the second IDE group.
